(19) United States
(12) Patent Application Publication
(10) Pub. No.: US 2024/0312005 A1
(43) Pub. Date: Sep. 19, 2024

(54) COMPUTER VISION DETECTION OF SKIN IRRITATION

(71) Applicant: KONINKLIJKE PHILIPS N.V., EINDHOVEN (NL)

(72) Inventors: Dietwig Jos Clement LOWET, Tongeren (BE); Jan Martijn Krans, EINDHOVEN (NL)

(21) Appl. No.: 18/443,807

(22) Filed: Feb. 16, 2024

Related U.S. Application Data
(60) Provisional application No. 63/452,809, filed on Mar. 17, 2023.

(51) Int. Cl. G06T 7/00; G06V 10/75; G06V 10/82

(52) U.S. Cl. CPC G06T 7/0012; G06V 10/751; G06V 10/82; G06T 2207/20084; G06T 2207/30088; G06V 2201/03

(57) ABSTRACT

An embodiment provides techniques for application of computer vision technology to medical images, allowing identification of skin irritation resulting from medical device wear. In certain embodiments, a subset of medical image data is identified, for example using template matching techniques or other trained computer vision model, facilitating timely and accurate identification of skin irritation, even at initial stages. An embodiment provides an application program that assists the user in obtaining and evaluating medical images, which may include instructing the user regarding any skin irritation identified, permitting remediation.

### BACKGROUND

### 1. Field

The disclosed subject matter generally pertains to application of computer vision to images for object identification and classification tasks. Certain disclosed subject matter relates to use of computer vision to address a problem with accurately detecting early-stage skin irritation associated with medical device wearing.

### 2. Description of the Related Art

Skin irritation is an important reason why people stop wearing a medical device that is adhered to the skin using an adhesive patch or sticker. For example, a Mobile Cardiac Outpatient Telemetry Patch System (“MCOT” or “MCOT patch”) is a monitor that adheres to the skin of the user in a chest region via a patch with adhesive. The MCOT patch gathers electrocardiogram (ECG) data from a sensor, then sends that ECG data to another device via a wireless connection such as BLUETOOTH, automatically. The MCOT patch is configured for long-term wear and will transmit cardiac or ECG data automatically 24 hours a day. By doing so throughout a monitoring period, the MCOT patch provides a healthcare professional with complete cardiac monitoring information.

It is important to be able to detect skin irritation associated with a medical device such as the MCOT patch as soon as possible to prevent deterioration of the skin condition and minimize patient discomfort. If skin irritation can be identified in a timely manner, an easy step to take is to place the medical device in a different location of the skin, such as a different location on the torso.

### SUMMARY

Identifying skin irritation due to medical device wearing at an early stage is challenging. Relying on manual or human review of medical images to classify skin irritation associated with medical device wearing is problematic as it requires expert reviewers and standardized image quality. Often manual review is only possible with a large or high degree of skin irritation, represented in high quality photos, without which a patient visit may be required, frustrating remote or automated analysis.

While conventional image processing techniques have been applied to examining skin for various conditions, these approaches have not been adapted to handle determination of skin irritation, particularly during early stages and when associated with medical device wearing. The need to refine computer vision applications to handle determination of skin irritation associated with medical device wearing is magnified when images are captured with varying quality, for example by non-expert users or patients under different conditions, with nonuniform hardware systems, such as various cameras available on smartphones.

Accordingly, an embodiment provides techniques for application of computer vision technology to medical images, allowing identification of skin irritation resulting from medical device wear, even at early stages where the signal of skin irritation in the images is not strong and/or when noisy images of varying quality are available. In certain embodiments, a subset of medical image data is identified, for example using template matching techniques or other trained computer vision model, facilitating timely and accurate identification of skin irritation, even at initial stages. An embodiment provides an application program that assists the user in obtaining and evaluating medical images, which may include instructing the user regarding any skin irritation identified, permitting remediation.

In summary, an embodiment provides a method comprising obtaining a medical image including a geometric shape associated with a medical device. The method includes analyzing, using a set of one or more processors, the medical image to identify the geometric shape and identifying a subset of image data of the medical image associated with the geometric shape. The method includes determining, using the set of one or more processors, that the subset of image data indicates skin irritation, and providing an indication of skin irritation.

In an embodiment, the method may include providing an instruction to capture the medical image, thereafter indicating the geometric shape within the medical image, and obtaining a confirmation that the geometric shape has been identified.

In an embodiment, the analyzing the medical image comprises performing template matching to identify the geometric shape, where the geometric shape comprises a predetermined patch shape.

In an embodiment, the analyzing the medical image comprises using a neural network to perform one or more of identifying the geometric shape and identifying the subset of image data associated with the geometric shape. In an embodiment, a trained model may perform the analyzing and identifying in a single classification step.

In an embodiment, the subset of image data associated with the geometric shape comprises one or more of pixel data located within a predetermined distance of the geometric shape and pixel data within the geometric shape.

In an embodiment, the determining the subset of image data indicates skin irritation comprises comparing one or more pixel values to a set of thresholds indicative of skin irritation. In an embodiment, the determining the subset of image data indicates skin irritation comprises directly classifying the medical image as including the predetermined shape and concluding that the subset of image data associated therewith indicates skin irritation.

In an embodiment, the method includes selecting an instruction based on the comparing the one or more pixel values of the subset of image data to the set of thresholds. In an embodiment, the instruction is selected based on a threshold from the set of thresholds, where the providing comprises including the instruction with the indication of skin irritation. In an embodiment, the instruction comprises one or more of audio data and visual data indicating that the medical device should be repositioned.

In an embodiment, obtaining of the medical image includes obtaining a first medical image prior to removal of the medical device, and thereafter obtaining the medical image after removal of the medical device, where the first medical image facilitates identification of the geometric shape in the medical image.

In an embodiment, the obtaining, the analyzing, the identifying, the determining, and the providing are performed locally on a client device.

An embodiment includes a computer program product comprising a non-transitory computer readable medium comprising code executable by a set of one or more processors. In an embodiment, the code comprises code that obtains a medical image comprising a geometric shape associated with a medical device, code that analyzes the medical image to identify the geometric shape, code that identifies a subset of image data of the medical image associated with the geometric shape, code that determines that the subset of image data indicates skin irritation, and code that provides an indication of skin irritation.

An embodiment includes a device, such as a user client device or a server offering a downloadable or executable image analysis program. In an embodiment, the device comprises a set of one or more processors and a non-transitory computer readable medium comprising code executable by the set of one or more processors. In an embodiment, the code comprises code that obtains a medical image comprising a geometric shape associated with a medical device, code that analyzes the medical image to identify the geometric shape, code that identifies a subset of image data of the medical image associated with the geometric shape, code that determines that the subset of image data indicates skin irritation, and code that provides an indication of skin irritation.

As will become apparent from reviewing this specification, methods, devices, systems, and products are provided for implementing the various embodiments. The foregoing is a summary and thus may contain simplifications, generalizations, and omissions of detail; consequently, those skilled in the art will appreciate that the summary is illustrative only and is not intended to be in any way limiting.

These and other features and characteristics of the example embodiments, as well as the methods of operation and functions of the related elements of structure and the combination thereof, will become more apparent upon consideration of the following description and the appended claims with reference to the accompanying drawings, all of which form a part of this specification. It is to be expressly understood, however, that the drawings are for the purpose of illustration and description only and are not intended as a definition of the limits of the invention.

### BRIEF DESCRIPTION OF THE DRAWINGS

FIG. 1 illustrates an example method according to an embodiment.

FIG. 2 illustrates an example of a geometric shape associated with a medical device and indicating skin irritation in a medical image according to an embodiment.

FIG. 3A illustrates an example medical image having a geometric shape associated with a medical device according to an embodiment.

FIG. 3B illustrates an example medical image having a geometric shape associated with a medical device and indicating skin irritation according to an embodiment.

FIG. 4A illustrates an example medical image having a geometric shape associated with a medical device according to an embodiment.

FIG. 4B illustrates an example medical image having a geometric shape associated with a medical device and indicating skin irritation according to an embodiment.

FIG. 5 illustrates a diagram of example system components according to an embodiment.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, e.g., through one or more intermediate parts or components, so long as a link occurs. As used herein, “operatively coupled” means that two or more elements are coupled so as to operate together or are in communication, unidirectional or bidirectional, with one another. As used herein, the term “number” shall mean one or an integer greater than one (i.e., a plurality). As used herein a “set” shall mean one or more.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well known structures, materials, or operations are not shown or described in detail to avoid obfuscation.

Medical devices (patches, monitors, sensors, and the like) may be adhered to a patient's skin via an adhesive containing portion, such as a patch, sticker, or tape. In certain circumstances, such as medical devices that are worn over a long period of time (e.g., hours or days), it is common for certain patients to experience skin irritation due to the wearing of the medical device.

When skin irritation is detected in a timely manner, it is potentially straightforward and easy to manage, e.g., by relocation of the medical device. Unfortunately, frequently this is not possible. For many patients it is not easy to detect skin irritation themselves, particularly at early stages when the negative impact of medical device wearing might be more easily avoided by relocating it to another area of the skin. Users might feel a slight itch or sensation on the skin but nonetheless have to inspect it in the mirror and attempt to determine whether potential redness is from the adhesive used to secure the medical device to the skin. If so, a user must also determine if the irritation is of nature where moving the medical device is advisable.

While conventional computer vision techniques have been applied to the problem space of examining skin for various conditions, conventional computer vision techniques do not easily handle determination of whether some redness on the skin of the patient is classifiable into a target category, particularly with relevance to medical device wear. What is needed is an improved computer vision technique that allows for identification of skin irritation attributable to or associated with medical device wear. This will allow a user to identify potentially problematic skin irritation more quickly, earlier in the development of the condition, and without a need to frequently follow up with a healthcare profession for manual photo review and instructions.

As skin irritation is an important reason why people stop wearing a medical device such as an MCOT patch, an embodiment provides computer vision techniques that allow skin irritation to be detected as early as possible. This permits accurate and timely identification of skin irritation associated with medical device wear and avoids further patient aggravation associated with unnecessarily asking the user to re-locate the medical device to a different location on the skin.

In an embodiment, a camera, for example of a smart phone or other user device, is used to capture a medical image (which may be a still image, series of still images, or a video) and a computer vision program is used to detect early onset of irritation. In an embodiment, a known geometric shape of the adhesive or patch portion of the medical device is utilized to assist in distinguishing a pattern for a computer vision process to look for irritation in a subset of the medical image to identify the skin irritation.

The description now turns to the figures. The illustrated example embodiments will be best understood by reference to the figures. The following description is intended only by way of example, and simply illustrates certain example embodiments.

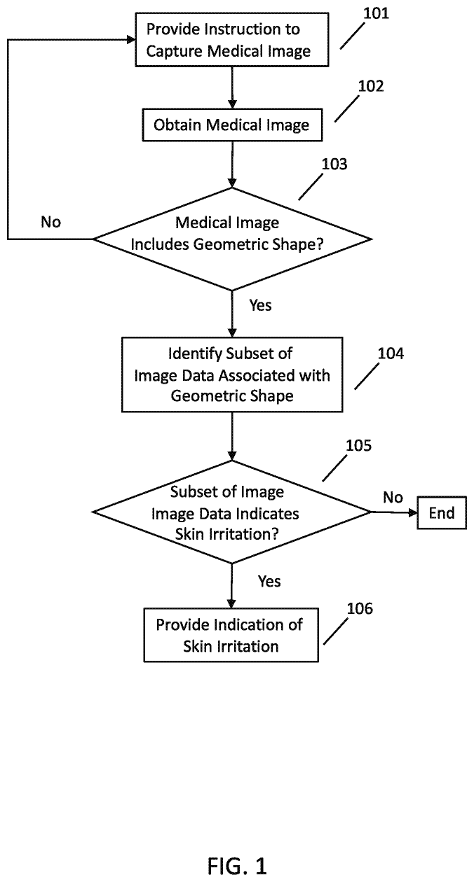

FIG. 1 illustrates an example method according to an embodiment. As illustrated, a method may include obtaining, at 102, a medical image comprising a geometric shape associated with a medical device. For example, the geometric shape may include one or more predetermined or expected shapes associated with the adhesive area or portion of a medical device such as an MCOT patch. In an embodiment, the medical image may be obtained as part of an application flow or sequence of instructions. For example, optionally the user may be instructed to capture a medical image including the medical device and the area or portion attached to the skin, as indicated at 101. As part of the application or sequence of instructions, an embodiment may provide feedback to the user as part of providing the instructions to capture the medical image at 101. For example, the user's device may be caused to display a visual or other indication, for example a bounding box, identifying a putative geometric shape. Such feedback provided may be based on the model(s) described herein used to identify the geometric shape as part of an object detection routine. Once the user has seen such feedback, and provided a confirmation, the image may be captured and/or obtained for use at 102.

The method may include analyzing, at 103, the medical image with model(s) to identify the geometric shape, such as the adhesive portion of the medical device attached to the user's skin. In one example, the analyzing at 103 may include identifying a predetermined shape, such as a geometric shape or outline of the adhesive portion of the medical device. In an embodiment indicating the geometric shape within the medical image that has been putatively identified and requesting a confirmation that the geometric shape has been correctly identified, after a confirmation that the geometric shape has been identified, the process may proceed with the analysis to identify a subset of image data at 104. If a user confirmation is not forthcoming or is negative, an embodiment may instruct the user to obtain another medical image, provide feedback regarding image settings or conditions preventing identification of the geometric shape, etc.

The analyzing of 103 may include performing template matching to identify the geometric shape, such as use of a template matching process available from OpenCV. As will be understood by those having ordinary skill in the art, an embodiment therefore makes use of the knowledge of a shape, such as a predetermined geometric shape of the MCOT patch, to perform object recognition within the medical image at 103. A template matching process may for example focus on detecting skin redness and/or skin irritation of a specific shape, for example the shape of the patch or a shape associated with the patch, such as a halo shape surrounding the periphery of the patch. This allows a computer vision program to detect skin irritation at a much earlier state, where an object comprising a subset of the medical image can become the focus of a classification task.

In an embodiment, if the patient is instructed to take a picture (or a short video clip) before removing the patch, this assists the computer vision program to utilize the initial image to readily identify an object (e.g., the medical device or portion thereof, such as the adhesive portion) and to know where on the skin the patch was located to subsequently identify the geometric shape in further images or video frames where the medical device has been removed. That is, in a session or sequence of an application or program, the user instruction provided at 101 may include a request to capture a medical image with the device being worn, and thereafter to capture a subsequent medical image with the medical device removed. Such instructions may include timing information, for example instructing the user to wait a period of time, e.g., several minutes, between image captures. After the medical device is removed, the system can focus on the subarea of the subsequent medical image, and more easily detect whether that area is more reddish or qualitatively different than the surrounding skin area, e.g., by comparison of image pixel values such as color content to a threshold, making a relative comparison (within and without a bounded area in an image, such as a subset of pixels associated with the adhesive portion of the medical device), or a combination of the foregoing.

An embodiment may utilize, at 103, a trained model such as a trained deep learning network to detect image characteristics, for example to perform object detection in the form of one or more colored patches of a certain shape contained within the medical image. A suitable neural network for this type of segmentation task is so-called U-NETs that are characterized by wide layers at the beginning and end and an information bottleneck in the middle. Using a noisy training set, such as the geometric shape of interest placed on noisy backgrounds, increases the susceptibility and precision of neural networks to detect shapes of a certain type as an object of interest, even when the signal is very weak, as may be the case during the initial stages of skin irritation. In this regard, in an embodiment the determination that the medical image includes the geometric shape 103, the identification of a subset of image data associated with the geometric shape at 104, and the determination that the subset of image data associated with the geometric shape indicates skin irritation at 105 may be a single classification task performed by a trained model to distinguish between normal or acceptable skin and skin irritation formed in a predetermined pattern of the geometric shape associated with the medical device.

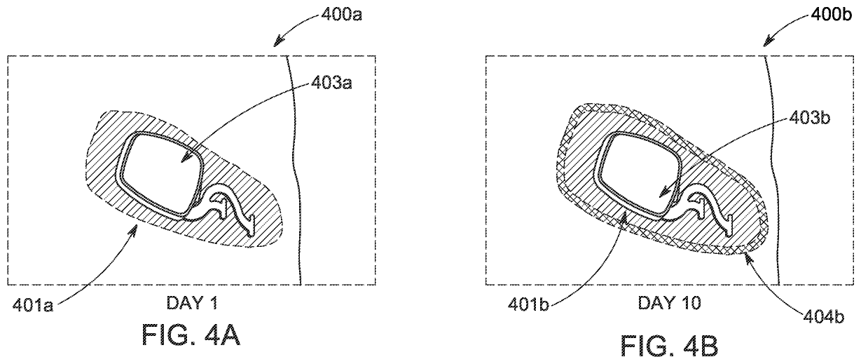

In an embodiment, the analyzing at 103 and identifying of a geometric shape facilitates identifying, at 104, a subset of image data of the medical image associated with the geometric shape. As described further herein, this may include identifying pixels of the medical image associated with the outline of the geometric shape. In one example, the identifying at 104 marks as set of pixels that lie within the outline or boundary defined by the geometric shape, as described in connection with FIG. 3B. In another example, the identifying at 104 marks a set of pixels that lie about the outline of the geometric shape, such as within a threshold distance of the outer margin of the geometric shape, as described in connection with FIG. 4B. In an embodiment, the identification or marking of the subset of data associated with the geometric shape may be used to provide feedback to the user, for example highlighting the area within the medical image identified as being relevant for analysis with respect to skin irritation associated with wearing of the medical device.

The method may include determining, at 105, that the subset of image data indicates skin irritation. As described herein, it is possible to utilize a model trained to identify the geometric shape itself and use such detection as a proxy for identification of skin irritation. In such an example, one or more models may be trained to identify the geometric shape and classify it, for example distinguishing classes of geometric shapes based on redness or color data.

An embodiment may also or alternatively further process the subset of image data associated with the geometric shape to identify and/or refine a type or characteristic of the skin irritation. For example, the pixels identified as associated with the geometric shape as a result of processing at steps 103 and/or 104 may be further analyzed at 105 to identify a skin irritation condition or classification based on the subset of image data, such as through additional comparison to one or more thresholds. In an example, the determining at 105 may include comparing the subset of image data to one or more thresholds to facilitate indicating that skin irritation of a certain type, level or character has been identified. In an embodiment, the one or more thresholds may be based on image data such as pixel color values, relative difference to other part(s) of the medical image, or similar.

As illustrated in FIG. 1, the method may include providing, 106, an indication of skin irritation following identification thereof. For example, an embodiment may provide an output via a smart phone application or similar program to indicate that skin irritation has been identified. In one example, an embodiment may utilize the one or more thresholds to associate the skin irritation with a level, score, or similar, suitable for selecting feedback or advice for the user, including for example an instruction to relocate the medical device to a specified area or similar instruction.

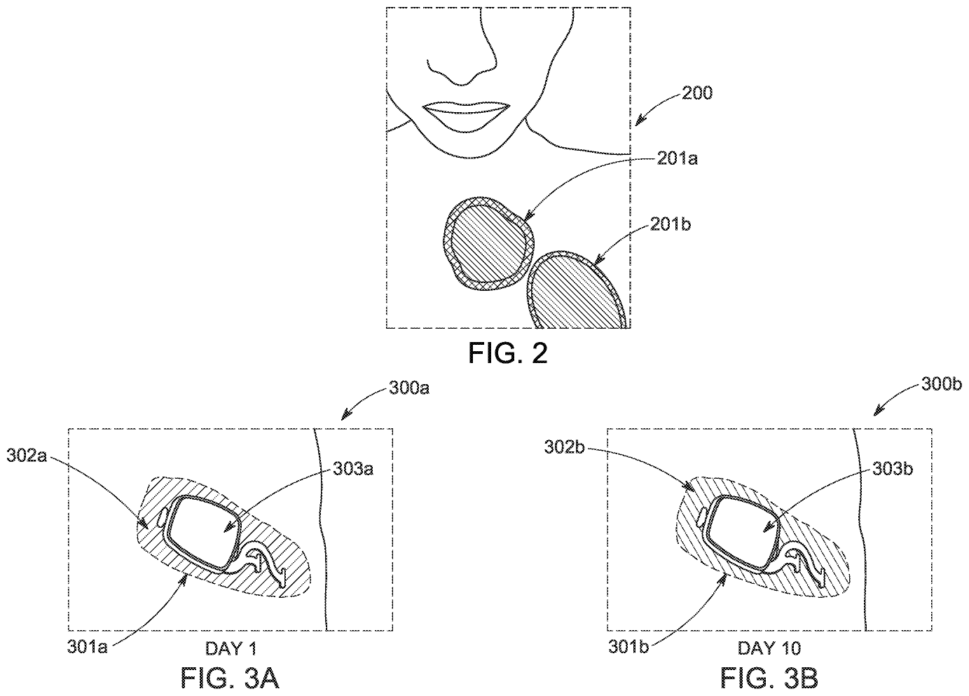

As shown in FIG. 2, a medical image 200 may include one or more geometric shape(s) 201a, 201b, associated with the adhesive portion (e.g., tape, patch, etc.) of a medical device worn on the skin of the user. In an embodiment, the computer vision process can be improved if the location of the geometric shape(s) 201a, 201b, are known, such as via performing a pre-processing step or steps that isolate a subset of image data. As described herein, this may be accomplished by taking a first medical image before removing the medical device and comparing it with medical image 200 when the medical device is removed. As described herein, the process may also include providing feedback regarding putative location(s) of the geometric shapes 201a, 201b within medical image 200 and receiving confirmation that the geometric shapes 201a, 201b have been properly identified. Visual markers in medical image 200 can be used to check whether any detected skin irritation is located at the place where the medical device was placed. Further, preprocessing to identify a subset of the medical image may be accomplished by applying one model to identify the subset, with subsequent applying of a second trained model or rule-based process to the subset for irritation detection or classification.

In an embodiment, referring to FIG. 3A-B, skin irritation may be detected by computer vision while the medical device 303a, 303b is still attached to the body. For example, if the adhesive portion (e.g., tape, patch, etc.) 301a, 301b of the medical device is transparent, visual changes to the skin often can be detected through adhesive 301a, 301b. By comparing medical images 300a, 300b of the patch on the body taken over time, for example over a ten-day period, differences in skin irritation in areas of the skin 302a, 302b associated with the geometric shape are detected by the system. In the example of FIG. 3A, medical image 300a shows medical device 303a (e.g., MCOT patch) with dotted lines indicated the edge or periphery of geometric shape defined by transparent adhesive 302a with which medical device 303a is mounted to the skin. In FIG. 3B, medical image 300b schematically shows irritation of the skin in area 302b (indicated for illustration in FIG. 3B with a different hatching as compared to 302a), underneath the transparent adhesive 301b, which will be detected by computer vision with medical device 303b still being mounted on the user.

As shown in FIG. 4A-B, in case of a non-transparent adhesive or tape, 401a, 401b, computer vision may be used to detect skin irritation area(s) 404b in medical image 400a, 400b over time while medical device 403a, 403b is still mounted on the body. That is, in case the skin irritation not only happens of the skin within an area covered by geometric shape of adhesive 401a, 401b, i.e., underneath adhesive 401a, 401b, but also or alternatively spreads to the skin proximate to adhesive 401a, 401b, as indicated in area 404b, this pattern, such as a halo effect, can be detected by obtaining medical images 400a, 400b including medical device 403a, 403b mounted on the body and having the computer vision process compare medical images 400a, 400b of medical device 403a, 403b taken on different days, for example on day 1 and day 10, to identify the geometric shape, which may be the halo effect area of medical image 400b, and make a determination regarding skin irritation, such as presence, type, level, recommended remediation, etc. Further, when time series data is available, it may be used to augment indication(s) or instruction(s), e.g., provided at 106 of FIG. 1, for example indicating a rate of irritation increase, predicted trend for skin irritation, etc.

In an embodiment, indication(s) or instruction(s), e.g., provided at 106 of FIG. 1, may include a prediction, such as an estimation of time remaining until skin irritation will be at a level requiring replacement action by the patient, such as movement of the patch to a new location. By way of example, medical image 400a (e.g., taken on day 1) may show irritation level 0, whereas medical image 400b (e.g., taken on day 2) may show irritation level 1. Accordingly, an embodiment, e.g., based on trend data for this patient and/or a patient population, can estimate at which future moment, day or time period the skin irritation will be at the level requiring replacement action and provide timely warning and/or further instruction(s).

In an embodiment, the indication(s) or instruction(s), e.g., provided at 106 of FIG. 1, may include a proposed placement area for a patch or medical device based on detected skin irritation area or subarea. For example, in a case of skin irritation, the action to be taken is to replace or move the patch. But in the case of an ECG patch, it cannot be placed just anywhere on non-irritated skin because it needs to be at a position at which the ECG signal still can be detected. Therefore, an ECG patch generally should not be shifted over too large of a distance. In a situation where skin irritation is not homogeneous over the full patch surface, as detected using computer vision supplied by an embodiment, a user may be instructed to avoid an area of irritation that is more prominent as compared to another area covered by the patch adhesive. Thus, an instruction provided at 106 may include advice to replace the patch to specifically avoid that subarea exhibiting increased irritation in medical image(s). As described herein, an embodiment may provide such instruction in various forms, including real-time augmented reality feedback, such as placing an icon or representation of the patch at the new location of the user skin, overlaid on a live video of the user captured using the patient's local device such as a smart phone or similar.

In the example of FIG. 4A-B, medical image 400a is obtained, e.g., by capturing via an application running on a user's smartphone, on a first day. The image is subject to analysis to identify a predetermined geometric shape, herein adhesive 401a within the image, associated with medical device 403a. Medical image 400a may be classified as not indicating skin irritation. Thereafter, for example according to a schedule of an application flow or program, another medical image 400b is obtained by the user. Medical image 400b is also analyzed to identify a predetermined shape, in this example one or more of adhesive 401b associated with medical device 403b or halo effect region indicated at area 404b. Area 404b in medical image 400b is identified as a subset of image data, i.e., the skin area surrounding a first geometric shape within a predetermined amount, such as a number of pixels, etc., or the skin area within a second geometric shape, i.e., the halo effect area 404b itself. Area 404b may then be utilized to determine if skin irritation is detected, e.g., surrounding adhesive 401b, either via direct indication based on a model's ability to detect area 404b as an object within medical image 400b (as compared to medical image 400a) and/or via submission of pixel data of area 404b to further analysis, such as comparison to one or more thresholds, further model classification or typing, and/or associating with instructions for feedback to the user.

An embodiment is therefore better able to detect skin irritation that is associated with a specific geometric shape, for example the known shape of the MCOT patch or a halo effect area associated therewith. An embodiment provides a mechanism to isolate a subset of image data for analysis of skin irritation, for example the subset of image data includes one or more of pixel data located within a predetermined distance of the geometric shape and pixel data within the geometric shape.

In an embodiment, the determining that the subset of image data indicates skin irritation includes comparing one or more pixel values to a set of thresholds indicative of skin irritation and provision of feedback for a user. For example, a data structure such as a table may store pixel value information, for example average or aggregate color values or ranges thereof, associated with instructions or program routines, for example instructions that display an indication of skin irritation, a request to move the medical device, etc. In an embodiment, an instruction is selected based on the comparing the one or more pixel values to the set of thresholds. By way of example, an aggregate pixel value for an area such as area 404b may fall within a numerical range indicative of initial skin irritation due to adhesive on the skin. The numerical range may be stored in a table or otherwise in logical association with an instruction, such as an instruction that skin irritation indicates that the medical device should be relocated or removed, e.g., for further imaging and analysis. In one example, the instruction may include one or more of audio data and visual data, for example, output via a mobile application, indicating that the medical device should be repositioned. The indication may further provide a visual or graphical display output that indicates a new position for the medical device, for example as part of an augmented reality program that places a suggested position for the medical device on an image of the user.

An embodiment may be implemented in a variety of devices, including user devices such as a smartphone or tablet running a mobile application. In one embodiment, referring back to FIG. 1, the obtaining 102, analyzing 103, identifying 104, determining 105, and providing 106 are performed locally on a client device such as a smartphone running a mobile application. In such an embodiment, a trained model such as a template matching model, a deep neural network, or similar, may be downloaded and run locally on the user device. For example, an application program, as described herein, may include an image analysis model, and include one or more application programming interfaces (APIs) to call a machine learning (ML) subsystem of the user device, for example calling image classification and/or object detection APIs of CORE ML on Apple's IPHONE. In some embodiments, the model version may be selected (e.g., downloaded, activated for running on an image, etc.) based on identification requirements, such as based on model or type of medical device, adhesive, and/or skin type. For example, a model trained to identify one or more predetermined shapes may be selected, for example by a user or as part of an application routine, based on knowledge of which type of device the user is wearing. Therefore, one or more models may be used in different circumstances, e.g., two or more models may be used, one for identifying each of two shapes of interest, such as medical devices and/or adhesive portions thereof having different geometric shapes, depending on which model of medical device the user is wearing.

Therefore, an embodiment may include an application program configured to execute computer program instructions, for example as outlined in FIG. 1, which in combination with local user device hardware, permit identification of geometric shapes of interest and classification of medical image data, for example the subset of medical image data associated with a geometric shape, to detect skin irritation and provide appropriate indication and feedback to the user. Such an embodiment permits the user's confidential medical images to remain solely on the user's device, increasing confidence of the user in applying any image analysis or computer vision techniques to the user's medical images.

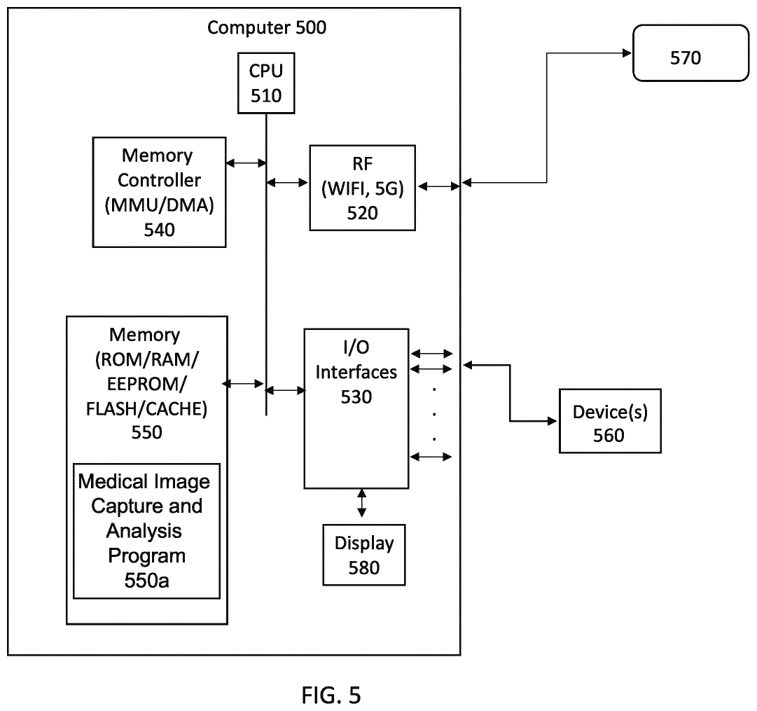

Referring to FIG. 5, it will be readily understood that certain embodiments can be implemented using any of a wide variety of devices or combinations of devices and components. In FIG. 5 an example of a computer 500 and its components are illustrated, which may be used in a device for implementing the functions or acts described herein, e.g., performing medical image analysis to identify skin irritation. Also, circuitry other than that illustrated in FIG. 5 may be utilized in one or more embodiments. The example of FIG. 5 includes certain functional blocks, as illustrated, which may be integrated onto a single semiconductor chip to meet specific application requirements.

One or more processing units are provided, which may include a central processing unit (CPU) 510, one or more graphics processing units (GPUs), and/or micro-processing units (MPUs), which include an arithmetic logic unit (ALU) that performs arithmetic and logic operations, instruction decoder that decodes instructions and provides information to a timing and control unit, as well as registers for temporary data storage. CPU 510 may comprise a single integrated circuit comprising several units, the design and arrangement of which vary according to the architecture chosen. As described herein, certain functional modules such as machine learning (ML) hardware or chip(s) may be included in a device such as computer 500 to facilitate certain functionality such as image analysis, object detection, object identification, etc.

Computer 500 also includes a memory controller 540, e.g., comprising a direct memory access (DMA) controller to transfer data between memory 550 and hardware peripherals. Memory controller 540 includes a memory management unit (MMU) that functions to handle cache control, memory protection, and virtual memory. Computer 500 may include controllers for communication using various communication protocols (e.g., I²C, USB, etc.).

Memory 550 may include a variety of memory types, volatile and nonvolatile, e.g., read only memory (ROM), random access memory (RAM), electrically erasable programmable read only memory (EEPROM), Flash memory, and cache memory. Memory 550 may include embedded programs, code and downloaded software, e.g., a medical image capture and analysis program 550a that provides coded methods such as illustrated and described in connection with FIG. 1. As described herein, program 550a may include a trained neural network trained using labeled training images (e.g., positive and/or negative examples of geometric shape(s), a training set providing geometric shape(s) of interest against noisy background image data, etc.) or descriptive metadata useful in identifying skin irritation based on classification thereof, as described herein. By way of example, and not limitation, memory 550 may also include an operating system, application programs, other program modules, code, and program data, which may be downloaded, updated, or modified via remote devices.

A system bus permits communication between various components of the computer 500. I/O interfaces 530 and radio frequency (RF) devices 520, e.g., WIFI and telecommunication radios, may be included to permit computer 500 to send data to and receive data from remote devices using wireless mechanisms, noting that data exchange interfaces for wired data exchange may be utilized. Computer 500 may operate in a networked or distributed environment using logical connections to one or more other remote computers or databases 570, such as a database storing trained models for analyzing medical images, databases storing downloadable applications or programs for the same, etc. The logical connections may include a network, such local area network (LAN) or a wide area network (WAN) but may also include other networks/buses. For example, computer 500 may communicate data with and between peripheral device(s) 560, for example a camera for capturing medical image data, which may be integrated within the same housing or unit as computer 500.

Computer 500 may therefore execute program instructions or code configured to obtain, store, and analyze medical image data and perform other functionality of the embodiments, such as described in connection with FIG. 1. A user can interface with (for example, enter commands and information) the computer 500 through input devices, which may be connected to I/O interfaces 530. A display 580 or other type of output device may be connected to or integrated with the computer 500, for example via an interface selected from I/O interfaces 530.

It should be noted that the various functions described herein may be implemented using instructions or code stored on a memory, e.g., memory 550, that are transmitted to and executed by a processor, e.g., CPU 510. Computer 500 includes one or more storage devices that persistently store programs and other data. A storage device, as used herein, is a non-transitory computer readable storage medium. Some examples of a non-transitory storage device or computer readable storage medium include, but are not limited to, storage integral to computer 500, such as memory 550, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations according to various embodiments may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections or through a hard wire connection, such as over a USB connection.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination. The word “about” or similar relative term as applied to numbers includes ordinary (conventional) rounding of the number with a fixed base such as 5 or 10.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method, comprising:
obtaining a medical image comprising a geometric shape associated with a medical device;
analyzing, using a set of one or more processors, the medical image to identify the geometric shape;
identifying, using the set of one or more processors, a subset of image data of the medical image associated with the geometric shape;
determining, using the set of one or more processors, that the subset of image data indicates skin irritation; and
providing, using the set of one or more processors, an indication of skin irritation.

2. The method of claim 1, comprising:
providing an instruction to capture the medical image;
thereafter indicating the geometric shape within the medical image; and
obtaining a confirmation that the geometric shape has been identified.

3. The method of claim 1, wherein the analyzing comprises performing template matching to identify the geometric shape.

4. The method of claim 3, wherein the geometric shape comprises a predetermined patch shape.

5. The method of claim 1, wherein one or more of the analyzing and the identifying comprises using a neural network to identify one or more of the geometric shape and the subset of image data associated with the geometric shape.

6. The method of claim 1, wherein the subset of image data associated with the geometric shape comprises one or more of pixel data located within a predetermined distance of the geometric shape and pixel data within the geometric shape.

7. The method of claim 1, wherein the determining comprises comparing one or more pixel values to a set of thresholds indicative of skin irritation.

8. The method of claim 7, comprising selecting an instruction based on the comparing the one or more pixel values to the set of thresholds.

9. The method of claim 9, wherein the instruction is selected based on a threshold from the set of thresholds.

10. The method of claim 9, wherein the providing comprises including the instruction with the indication of skin irritation.

11. The method of claim 10, wherein the instruction comprises one or more of audio data and visual data indicating that the medical device should be repositioned.

12. The method of claim 1, wherein the obtaining comprises:
obtaining a first medical image prior to removal of the medical device; and
thereafter obtaining the medical image after removal of the medical device;
wherein the first medical image facilitates identification of the geometric shape in the medical image.

13. The method of claim 1, wherein the obtaining, the analyzing, the identifying, the determining, and the providing are performed locally on a client device.

14. A computer program product, comprising:
a non-transitory computer readable medium comprising code executable by a set of one or more processors, the code comprising:
code that obtains a medical image comprising a geometric shape associated with a medical device;
code that analyzes the medical image to identify the geometric shape;
code that identifies a subset of image data of the medical image associated with the geometric shape;
code that determines that the subset of image data indicates skin irritation; and
code that provides an indication of skin irritation.

15. A device, comprising:
a set of one or more processors; and
a non-transitory computer readable medium comprising code executable by the set of one or more processors, the code comprising:
code that obtains a medical image comprising a geometric shape associated with a medical device;
code that analyzes the medical image to identify the geometric shape;
code that identifies a subset of image data of the medical image associated with the geometric shape;
code that determines that the subset of image data indicates skin irritation; and
code that provides an indication of skin irritation.
